SEQUENCE LISTING (18 sequences)

SEQ ID NO: 1 - DNA, 200 bp, artificial sequence
  the target gRNA-1
gctggagcagctggagatggccatggggatgggcggcgtgagcgcccccggcgccgcggatgacgggttcgtgtcgcacctggccacggacaccgtgcactacaacccctcggacctctcctcctgggtcgagagcatgctttccgagctcaacgcgccgctgccccctatcccgccagcgccgccggctgcccgccatg

SEQ ID NO: 2 - DNA, 347 bp, artificial sequence
  the OSU6-gRNA-1 sequence
ggatcatgaaccaacggcctggctgtatttggtggttgtgtagggagatggggagaagaaaagcccgattctcttcgctgtgatgggctggatgcatgcgggggagcgggaggcccaagtacgtgcacggtgagcggcccacagggcgagtgtgagcgcgagaggcgggaggaacagtttagtaccacattgcccagctaactcgaacgcgaccaacttataaacccgcgcgctgtcgcttgtgtgacccaggaggagaggtccggttttagagctagaaatagcaagttaaaataaggctagtccgttatcaacttgaaaaagtggcaccgagtcggtgctttttt

SEQ ID NO: 3 - DNA, 96 bp, artificial sequence
  single-stranded oligonucleotide fragments
aattcagttgggtcgagagcatgctttccgagctcaacgcgccgctgccccctatcccgccagcgccgccggctgcccgccatgcttccacctcgg

SEQ ID NO: 4 - DNA, 96 bp, artificial sequence
  single-stranded oligonucleotide fragments
ccgaggtggaagcatggcgggcagccggcggcgctggcgggatagggggcagcggcgcgttgagctcggaaagcatgctctcgacccaactgaatt

SEQ ID NO: 5 - DNA, 120 bp, artificial sequence
  single-stranded oligonucleotide fragments
cgcggatgacgggttcgtgtcgcacctggccacggacaccgtgcactacaacccctcggaattcagttgggtcgagagcatgctttccgagctcaacgcgccgctgccccctatcccgcc

SEQ ID NO: 6 - DNA, 18 bp, artificial sequence
  primer
gcgctcgggtacaaggtg

SEQ ID NO: 7 - DNA, 19 bp, artificial sequence
  primer
ccagcctcctgcgtgtcaa

SEQ ID NO: 8 - DNA, 20 bp, artificial sequence
  primer
gctctcgacccaactgaatt

SEQ ID NO: 9 - DNA, 300 bp, artificial sequence
  target gRNA-2
tcctgaaaggaagtacagtgtctggattggaggatccatcttggcatctctcagcacattccagcaggtaaatatacaaatgcagcaatgtagtgtgtttacctcatgaacttgatcaatttgcttacaatgttgcttgccgttgcagatgtggattgccaaggctgagtacgacgagtctggcccatccattgtgcacaggaaatgcttctaattcttcggacccaagaatgctaagccaagaggagctgttatcgccgtcctcctgcttgtttctctctttttgttgctgtttcttca

SEQ ID NO: 10 - DNA, 201 bp, artificial sequence
  target gRNA-3
ttatttctaaaacacattcagaatcaaatcattgggttctgtattgttgttgtgcaggctctgtttagccgcgaatctttcatcaagacgaaggcagctaaggagcacctgattgctggatgggcaccaaaagtgaatgcgtaagagcctgcccttatgctctggtgctgcttggacaccatgctgtttatctgatcggtc

SEQ ID NO: 11 - DNA, 347 bp, artificial sequence
  OSU6-gRNA- 2
ggatcatgaaccaacggcctggctgtatttggtggttgtgtagggagatggggagaagaaaagcccgattctcttcgctgtgatgggctggatgcatgcgggggagcgggaggcccaagtacgtgcacggtgagcggcccacagggcgagtgtgagcgcgagaggcgggaggaacagtttagtaccacattgcccagctaactcgaacgcgaccaacttataaacccgcgcgctgtcgcttgtgtgtggcccatccattgtgcacgttttagagctagaaatagcaagttaaaataaggctagtccgttatcaacttgaaaaagtggcaccgagtcggtgctttttt

SEQ ID NO: 12 - DNA, 347 bp, artificial sequence
  OSU6-gRNA-3
ggatcatgaaccaacggcctggctgtatttggtggttgtgtagggagatggggagaagaaaagcccgattctcttcgctgtgatgggctggatgcatgcgggggagcgggaggcccaagtacgtgcacggtgagcggcccacagggcgagtgtgagcgcgagaggcgggaggaacagtttagtaccacattgcccagctaactcgaacgcgaccaacttataaacccgcgcgctgtcgcttgtgtgggagcacctgattgctggagttttagagctagaaatagcaagttaaaataaggctagtccgttatcaacttgaaaaagtggcaccgagtcggtgctttttt

SEQ ID NO: 13 - DNA, 30 bp, artificial sequence
  primer
ctactcgagggggtggcccatccattgtgc

SEQ ID NO: 14 - DNA, 32 bp, artificial sequence
  primer
gatgactactcgaggagcacctgattgctgga

SEQ ID NO: 15 - DNA, 32 bp, artificial sequence
  primer
gtggatccatcgttcaaacatttggcaataaa

SEQ ID NO: 16 - DNA, 20 bp, artificial sequence
  primer
tgtgagggacatgaaggaga

SEQ ID NO: 17 - DNA, 20 bp, artificial sequence
  primer
gtacacttgtcctgtctgga

SEQ ID NO: 18 - DNA, 20 bp, artificial sequence
  primer
acgctgaacttgtggccgtt

(19) United States
(12) Patent Application Publication
(10) Pub. No.: US 2021/0095299 A1
(43) Pub. Date: Apr. 1, 2021

(54) REPEAT-MEDIATED PLANT SITE-SPECIFIC RECOMBINATION METHOD

(71) Applicant: CAS CENTER FOR EXCELLENCE IN MOLECULAR PLANT SCIENCES, Shanghai (CN)

(72) Inventors: Jiankang ZHU, Shanghai (CN); Yuming LU, Shanghai (CN); Yifu TIAN, Shanghai (CN); Rundong SHEN, Shanghai (CN); Mugui WANG, Shanghai (CN)

(21) Appl. No.: 17/050,168

(22) PCT Filed: Apr. 10, 2019
(86) PCT No.: PCT/CN2019/082095

§ 371 Date: Oct. 23, 2020

(30) Foreign Application Priority Data
Apr. 23, 2018 (CN) 201810368574.2

(51) Int. Cl. C12N 15/82; C12N 9/22; C12N 15/11

(52) U.S. Cl. CPC C12N 15/8213; C12N 15/11; C12N 9/22

(57) ABSTRACT

Provided is a donor DNA having a specific repeated sequence, and a reagent kit for gene editing. Also provided is a repeat-mediated plant site-specific recombination method, comprising using the donor DNA having the specific repeated sequence, cleaving a specific site of a target gene using a site-specific cleaving nuclease, and integrating the donor DNA fragment into a cleavage site by using a homologous arm of the donor DNA.

### TECHNICAL FIELD

The present invention relates to the field of biotechnology, in particular, to a site-specific recombination method in plant mediated by repeat fragments.

### BACKGROUND

Genome editing technologies include Zinc finger nuclease (ZFN), transcription activator-like (TAL) effector nucleases (Talen) and CRISPR/Cas technology. All these three technologies can cleave DNA specifically to produce double-strand breaks (DSB) at specific sites in the organism's genome, and the non-homologous end joining or homologous recombination characteristic of the cell itself can be used for site-specific editing. ZFN and Talen technologies use specific proteins to guide genome cutting, but their construction is relatively complex and editing efficiency is very low.

It is generally believed that the DNA break repair mechanism in plant cells is dominated by NHEJ, and the probability of HDR is relatively very low. Therefore, when performing genome editing, it is mainly based on the results after NHEJ repair. Although site-specific knock-in or replacement can also be performed through the NHEJ pathway, the editing result is shown as Indel of the target position, and the recombination efficiency of NHEJ in rice is very low, the highest is only about 2%. At present, it has always been a problem in the field of plants to achieve precise knock-in through HDR. Unless the knock-in or replacement sequence is a screening tag, otherwise, its implementation efficiency is very low. So far, there has been a lack of efficient methods for precise genome knock-in/replacement in the plant field.

In conclusion, for the needs of plant research and breeding, there is an urgent need to develop an efficient genome precision knock-in/replacement technology in the art.

### SUMMARY OF THE INVENTION

The purpose of the present invention is to provide an efficient genome precision knock-in/replacement technology.

In a first aspect of the present invention, it provides a nucleic acid construct having a structure as shown in Formula I from 5′-3′:

Y1-Z1-Z2-Z3-Z4-Z5-Y2 (I)

wherein Y1 is none or a nucleotide sequence;
Z1 is a first DSB sequence;
Z2 is a first homologous sequence;
Z3 is a target DNA sequence;
Z4 is a second homologous sequence;
Z5 is a second DSB sequence;
Y2 is none or a nucleotide sequence;

and each “-” is independently a bond or a nucleotide linking sequence.

In another preferred embodiment, the nucleotide linking sequence comprises a sequence of m nucleotides in length, wherein m is 1-30, preferably 1-20, more preferably 1-10 (such as 1, 2, 3, 4, 5, 6, 7, 8, 9, 10).

In another preferred embodiment, each “-” is a bond.

In another preferred embodiment, the first DSB sequence and the second DSB sequence are located (identified) and cleaved with the participation of gRNA.

In another preferred embodiment, each DSB sequence can be recognized and cleaved by a site-directed cleaving nuclease.

In another preferred embodiment, each of the DSB sequences is independently:

(a) containing a cleavage site itself, or (b) forming a cleavage site after the nucleic acid construct is integrated into the target site by NHEJ.

In another preferred embodiment, the first DSB sequence may be outside the 5′end of the first homologous sequence.

In another preferred embodiment, the first DSB sequence partially overlaps with the first homologous sequence.

In another preferred embodiment, the second DSB sequence may be outside the 3′end of the second homologous sequence.

In another preferred embodiment, the second DSB sequence partially overlaps with the second homologous sequence.

In another preferred embodiment, the first DSB sequence and the second DSB sequence are the same or different.

In another preferred embodiment, the first DSB sequence and the second DSB sequence are the same or different from the DSB sequence of the cleavage site of the genome target site (“target site DSB sequence”).

In another preferred embodiment, the first DSB sequence, the second DSB sequence and the target site DSB sequence are the same.

In another preferred embodiment, the site-directed cleaving nuclease is selected from the group consisting of ZFN, Talen and CRISPR/Cas9, and a combination thereof.

In another preferred embodiment, the site-directed cleaving nuclease is CRISPR/Cas9.

In another preferred embodiment, the target DNA sequence can be recognized and cleaved by an enzyme selected from the group consisting of a CRISPR-related enzyme such as Cas9, Cpf1, C2C1, C2C2, and C2C3.

In another preferred embodiment, the target DNA sequence can be recognized and cleaved by an enzyme selected from the group consisting of Fok I.

In another preferred embodiment, the first DSB sequence is 10-50 bp, preferably 15-30 bp.

In another preferred embodiment, the second DSB sequence is 10-50 bp, preferably 15-30 bp.

In another preferred embodiment, the first homologous sequence is 20 bp-10 kb, preferably 30 bp-5 kb.

In another preferred embodiment, the second homologous sequence is 20 bp-10 kb, preferably 30 bp-5 kb.

In another preferred embodiment, the target DNA sequence is a sequence to be knocked in and/or replaced.

In another preferred embodiment, the target DNA sequence is lbp-10 kb, preferably 5 bp-5 kb.

In another preferred embodiment, the Y1 and Y2 are protective bases.

In another preferred embodiment, the lengths of Y1 and Y2 are respectively 1-50 bp, preferably 4-20 bp.

In another preferred embodiment, the first homologous sequence has sequence homology H1 with the DNA sequence on one side (such as the upstream side or the left side) of the target site of the eukaryotic cell genome, the second homologous sequence has homology H2 with the DNA sequence on the other side (such as the downstream side or the right side) of the target site of the eukaryotic cell genome, respectively, both H1 and H2 are ≥90%, more preferably, ≥95%.

In another preferred embodiment, the first homologous sequence, the second homologous sequence and the DNA sequence on both sides of the target site of the eukaryotic cell genome respectively constitute a direct repeat sequence (that is, the first homologous sequence and the DNA sequence on one side of the target site (such as the upstream side or the left side) constitute a direct repeat sequence, and the second homologous sequence and the DNA sequence on the other side of the target site (such as the downstream side or the right side) constitute a direct repeat sequence; vice versa).

In another preferred embodiment, the eukaryotic cell comprises a plant cell.

In another preferred embodiment, the plant comprises an angiosperm andgymnosperm.

In another preferred embodiment, the gymnosperm is selected from the group consisting of Cycadaceae, Podocarpaceae, Araucariaceae, Pinaceae, Taxodiaceae, Cupressaceae, Cephalotaxaceae, Taxaceae, Ephedraceae, Gnetaceae, monotypic family, Welwitschiaceae, and a combination thereof.

In another preferred embodiment, the plant comprises a monocotyledon and dicotyledon.

In another preferred embodiment, the plant comprises a herbaceous plant and woody plant.

In another preferred embodiment, the herbaceous plant is selected from the group consisting of Solanaceae, Gramineous plants, Leguminous plants, and a combination thereof.

In another preferred embodiment, the woody plant is selected from the group consisting of Actinidiaceae, Rosaceae, Moraceae, and a combination thereof.

In another preferred embodiment, the plant is selected from the group consisting of Cruciferous plants, Gramineous plants, Leguminous plants, Solanaceae, Actinidiaceae, Malvaceae, Paeoniaceae, Rosaceae, Liliaceae, and a combination thereof.

In another preferred embodiment, the plant is selected from the group consisting of rice, Chinese cabbage, soybean, tomato, corn, tobacco, wheat, sorghum and a combination thereof.

In another preferred embodiment, the nucleic acid construct is a single-stranded DNA sequence or a double-stranded DNA sequence, preferably a double-stranded DNA sequence.

In another preferred embodiment, the 5′ end(s) of one and/or two DNA single strand(s) of the nucleic acid construct are phosphorylated.

In another preferred embodiment, two 5′ ends of the two DNA single strands of the nucleic acid construct are both phosphorylated.

In another preferred embodiment, the phosphodiester bond between one or more (such as 2, 3, 4, or 5) bases at the end of 5′ and/or 3′ end of the nucleic acid construct is thio modified.

In another preferred embodiment, there is no screening tag on the nucleic acid construct.

In a second aspect of the present invention, it provides a reagent combination for gene editing, comprising:

(i) a first nucleic acid construct, or a first vector containing the first nucleic acid construct, the first nucleic acid construct has a structure of Formula I from 5′-3′:

P1-A1-A2 (I)

wherein P1 is a first promoter;
A1 is a coding sequence of Cas9 protein;
A2 is a terminator;

and, “-” is a bond or a nucleotide linking sequence; and

(ii) a donor DNA element, the donor DNA element comprises: the nucleic acid construct according to the first aspect of the present invention, or a vector for expressing the nucleic acid construct.

In another preferred embodiment, the donor DNA comprises: a second nucleic acid construct, or a second vector containing the second nucleic acid construct.

In another preferred embodiment, the second nucleic acid construct has a structure as shown in Formula II from 5′-3′:

P2-A3-A4-A5 (II)

wherein P2 is a second promoter;
A3 is a coding sequence of gRNA;
A4 is none or a transcription termination sequence;
A5 is an expression cassette of the nucleic acid construct of claim 1;

and, “-” is a bond or a nucleotide linking sequence.

In another preferred embodiment, the nucleotide linking sequence is 1-60 nt.

In another preferred embodiment, the nucleotide linking sequence does not affect the normal transcription and translation of each element.

In another preferred embodiment, the first promoter comprises a PolII promoter.

In another preferred embodiment, the first promoter is selected from the group consisting of 35S promoter, UBQ promoter, Actin promoter, UBI promoter, and a combination thereof.

In another preferred embodiment, the second promoter comprises a PolII promoter.

In another preferred embodiment, the second promoter is selected from the group consisting of tRNA promoter, 35S promoter, UBQ promoter, Actin promoter, UBI promoter, and a combination thereof.

In another preferred embodiment, the tRNA promoter is selected from the group consisting of U6 promoter, U3 promoter, and a combination thereof.

In another preferred embodiment, the Cas9 protein is selected from the group consisting of: Cas9, Cas9n, and a combination thereof.

In another preferred embodiment, the source of the Cas9 protein is selected from the group consisting of Streptococcus pyogenes, Staphylococcus aureus, and a combination thereof.

In another preferred embodiment, the terminator is selected from the group consisting of: NOS terminator, UBQ terminator, and a combination thereof.

In another preferred embodiment, the transcription termination sequence is selected from the group consisting of PolyA, PolyT, NOS terminator, UBQ terminator, and a combination thereof.

In another preferred embodiment, the polyT sequence is Poly(T)ₙ, wherein n is 5-30.

In another preferred embodiment, the polyA sequence is Poly(A)ₙ, wherein n is 5-30.

In another preferred embodiment, the first vector and the second vector are different vectors.

In another preferred embodiment, the first nucleic acid construct and the second nucleic acid construct are located on different vectors.

In another preferred embodiment, the vector is a binary expression vector that can be transfected or transformed into plant cells.

In another preferred embodiment, the vector is a plant expression vector.

In another preferred embodiment, the vector is pCambia vector.

In another preferred embodiment, the plant expression vector is selected from the group consisting of pCambia1300, pCambia3301, pCambia2300, and a combination thereof.

In another preferred embodiment, the vector is Agrobacterium Ti vector.

In another preferred embodiment, the vector is circular or linear.

In another preferred embodiment, the plant comprises an angiosperm and gymnosperm.

In another preferred embodiment, the gymnosperm is selected from the group consisting of Cycadaceae, Podocarpaceae, Araucariaceae, Pinaceae, Taxodiaceae, Cupressaceae, Cephalotaxaceae, Taxaceae, Ephedraceae, Gnetaceae, monotypic family, Welwitschiaceae, and a combination thereof.

In another preferred embodiment, the plant comprises a monocotyledon and dicotyledon.

In another preferred embodiment, the plant comprises a herbaceous plant and woody plant.

In another preferred embodiment, the herbaceous plant is selected from the group consisting of Solanaceae, Gramineous plants, Leguminous plants, and a combination thereof.

In another preferred embodiment, the woody plant is selected from the group consisting of Actinidiaceae, Rosaceae, Moraceae, and a combination thereof.

In another preferred embodiment, the plant is selected from the group consisting of Cruciferous plants, Gramineous plants, Leguminous plants, Solanaceae, Actinidiaceae, Malvaceae, Paeoniaceae, Rosaceae, Liliaceae, and a combination thereof.

In another preferred embodiment, the plant is selected from the group consisting of rice, Chinese cabbage, soybean, tomato, corn, tobacco, wheat, sorghum and a combination thereof.

In another preferred embodiment, the gene editing is gene-directed knock-in and/or replacement.

In a third aspect of the present invention, it provides a kit containing the reagent combination according to the second aspect of the present invention.

In another preferred embodiment, the kit further contains a label or instructions.

In a fourth aspect of the present invention, it provides a method for gene editing of a plant or plant cell, which comprises: in the presence of a donor DNA, integrating the donor DNA into a target site of the plant cell genome through NHEJ, and then DSB cleavage is performed on the sequence from the donor DNA integrated into the target site, thereby performing homologous recombination (HDR) based on the homologous sequence, thereby site-directed introducing the target DNA sequence from the donor DNA at the target site.

In another preferred embodiment, the target DNA sequence comprises a single base, multiple bases, a nucleic acid fragment, or a single gene, or multiple genes.

In another preferred embodiment, the homologous recombination is based on the homology between the first homologous sequence of the target DNA sequence on the donor DNA and the homologous sequence on the upstream (or left) side of the target site, and the homology between the second homologous sequence of the target DNA sequence on the donor DNA and the homologous sequence on the downstream side (or right side) of the target site.

In another preferred embodiment, the method comprises the steps:
(a) providing a donor DNA and a plant to be edited, wherein the donor DNA has a structure as shown in Formula I from 5′-3′:

Y1-Z1-Z2-Z3-Z4-Z5-Y2 (I)
wherein Y1 is none or a nucleotide sequence;
   Z1 is a first DSB sequence;
   Z2 is a first homologous sequence;
   Z3 is a target DNA sequence;
   Z4 is a second homologous sequence;
   Z5 is a second DSB sequence;
   Y2 is none or a nucleotide sequence;
and each “-” is independently a bond or a nucleotide linking sequence;
(b) in the presence of the donor DNA, performing NHEJ and HDR on the plant to be edited successively, thereby realizing the editing of the target gene of the plant cell.

In another preferred embodiment, in step (b), after NHEJ and HDR, the homologous sequences on both sides of the target site of the plant cell are homologous sequences in the donor DNA.

In a fifth aspect of the present invention, it provides a method for gene-editing a plant or plant cell, comprising the steps:

(i) providing a plant or plant cell to be edited;

(ii) introducing a first nucleic acid construct or a first vector containing the first nucleic acid construct, and a donor DNA element comprising the nucleic acid construct according to the first aspect of the present invention, or the vector for expressing the nucleic acid construct into the plant cell of the plant to be edited, thereby realizing the editing of the target gene of the plant or plant cell;

wherein the first nucleic acid construct has a structure of Formula I from 5′-3′:

P1-A1-A2 (I)

wherein P1 is a first promoter;
A1 is a coding sequence of Cas9 protein;
A2 is a terminator;

and, “-” is a bond or a nucleotide linking sequence.

In another preferred embodiment, the donor DNA comprises: a second nucleic acid construct, or a second vector containing the second nucleic acid construct.

In another preferred embodiment, the second construct has a structure as shown in Formula II from 5′-3′:

P2-A3-A4-A5 (II)

wherein P2 is a second promoter;
A3 is a coding sequence of gRNA;
A4 is none or a transcription termination sequence;
A5 is an expression cassette of the nucleic acid construct of claim 1;

and, “-” is a bond or a nucleotide linking sequence.

In another preferred embodiment, the first vector and the second vector are introduced simultaneously or sequentially.

In another preferred embodiment, the introduction is performed by Agrobacterium.

In another preferred embodiment, the introduction is performed by gene gun.

In another preferred embodiment, the gene editing is site-directed knock-in and/or replacement.

In another preferred embodiment, the target gene contains a site recognized and cleaved by a site-directed cleavage nuclease.

In another preferred embodiment, when the first DSB sequence and the second DSB sequence in the A5 element are the same as the DSB sequence of the cleavage site of the genome target site (“target site DSB sequence”), the method is realized through one genetic transformation.

In another preferred embodiment, when the first DSB sequence and the second DSB sequence in the A5 element are not the same as the DSB sequence of the cleavage site of the genome target site (“target site DSB sequence”), the method can be achieved through two genetic transformations, or through one genetic transformation.

In a sixth aspect of the present invention, it provides a method for preparing a transgenic plant cell, comprising the steps:

(i) introducing or transfecting the nucleic acid construct according to the first aspect of the present invention or the reagent combination according to the second aspect of the present invention into a plant cell, so that the nucleic acid construct according to the first aspect of the present invention or the nucleic acid construct in the reagent combination according to the second aspect of the present invention and the chromosome in the plant cell undergo site-directed knock-in and/or replacement, thereby preparing the transgenic plant cell.

In another preferred embodiment, the transfection adopts the Agrobacterium transformation method or the gene gun bombardment method.

In a seventh aspect of the present invention, it provides a method for preparing a transgenic plant cell, comprising the steps:

(i) introducing or transfecting the nucleic acid construct according to the first aspect of the present invention or the reagent combination according to the second aspect of the present invention into a plant cell, so that the plant cell contains the nucleic acid construct according to the first aspect of the present invention or the construct in the reagent combination according to the second aspect of the present invention, thereby preparing the transgenic plant cell.

In an eighth aspect of the present invention, it provides a method for preparing a transgenic plant, comprising the steps:

Regenerating the transgenic plant cell prepared by the method according to the sixth aspect of the present invention or the seventh aspect of the present invention into a plant, thereby obtaining a transgenic plant.

In a ninth aspect of the present invention, it provides a transgenic plant cell prepared by the method according to the sixth aspect of the present invention or the seventh aspect of the present invention.

In a tenth aspect of the present invention, it provides a transgenic plant prepared by the method according to the eighth aspect of the present invention.

It should be understood that, within the scope of the present invention, each technical feature of the present invention described above and in the following (as examples) may be combined with each other to form a new or preferred technical solution, which is not listed here due to space limitations.

### DESCRIPTION OF FIGURE

FIG. 1 shows a schematic diagram of a genome-directed knock-in method mediated by repetitive fragments. (A) The donor DNA fragment used for site-directed knock-in/replacement contains the sequence to be replaced/knocked-in (101), both ends of which are 5′ phosphorylated modified (102), and have regions of homology with the genome sequence at the position to be knocked in (104), and there is a site-directed cleavage site outside the homologous region, or a site-directed cleavage site can be formed after site-specific inserting into the genome (103). The fragment can generally be synthesized or amplified by PCR with corresponding primers (106). (B) DSB is caused by nuclease targeted cleavage of the genomic site, (C) the donor DNA fragment is integrated into the target site through NHEJ; since the donor fragment contains a sequence homologous to the target site, it can form repeat sequences in the same direction, the site-specific cleavage sites existing or formed between the repeat sequences can produce DSB; because the DSB between the repeat sequences can produce very efficient HDR (105), (D) using this feature of the cell to achieve site-directed precise knock-in/replacement.

FIG. 2 shows a schematic diagram of a newly formed cleavage site. After the donor DNA fragment is site-directed inserted into the genome, a new cleavage site is formed (201).

FIG. 3 shows a schematic diagram of the base substitution of the rice SLR1 gene. A. Donor DNA fragment sequence and its structure, wherein 301 is the sequence to be replaced; 302 is the partial sequence of the newly formed CRISPR/Cas9 target gRNA-1 after site-specific insertion into the genome; and 303 is the sequence homologous to genomic DNA (underlined). B. The process of base substitution. 304 is the DNA sequence of the wild-type SLR1 target site; 305 is the target sequence that needs to be replaced; 306 is the CRISPR/Cas9 target gRNA-1; 307 is the donor DNA fragment (double-stranded); 308 is the newly formed CRISPR/Cas9 target gRNA-1 after the donor DNA is site-directed inserted into the genome, wherein the red partial sequence and the green partial sequence form a continuous repeat sequence.

FIG. 4 shows the results of base substitution detection of rice SLR1 gene. A. Schematic diagram of rice SLR1 gene; B. PCR detection result of T0 generation plant site-specific replacement; C. Sanger sequencing result of T0 generation plant #30; D. statistical table of SLR1 gene site-specific replacement efficiency; E. The phenotypes of the T0 generation plants, from left to right, are the phenotypes of plants with Indel mutation, site-specific replacement and wild type.

FIG. 5 shows the results of the site-specific integration of GFP in rice ACT1 and GST1. A. Schematic diagram of the integration of GFP into the 3′ ends of ACT1 and GST1; B. PCR detection results of T0 generation plants, wherein the upper part is the specific amplification result, and the lower part is the internal reference control; C. Sanger sequencing results of T0 generation plants ACT #01 and GST #03; E. statistics table of GFP targeted knock-in results.

### DETAILED DESCRIPTION OF INVENTION

After extensive and intensive research, the inventors have screened out a donor DNA with a specific repeat sequence structure through a large number of screenings, and cut the specific site of the target gene with the help of a site-directed cleavage nuclease to integrate the donor DNA fragment into the cleavage site.

The donor DNA of the present invention has a sequence homologous to the target gene sequence of the genome, and the donor DNA has one or more site-directed cleavage sites outside of the homologous sequence or in the region overlapping with the homologous sequence, or a site-directed cleavage site will be formed after site-specific insertion into the genome. Since the donor fragment contains a sequence homologous to the target site, it can form repetitive sequences in the same direction. The site-directed cleavage site that exists or formed between the repetitive sequences can produce DSB, thereby producing very efficient HDR, which further realizes efficient site-specific knock-in and/or replacement, and further experiments have shown that modifying the donor DNA fragment can efficiently integrate the donor DNA fragment into the genome of the recipient plant, with an efficiency of ≥12%. The recombination efficiency is improved by more than 6 times, compared with traditional methods (only NHEJ or HDR). On this basis, the inventors have completed the present invention.

### Terms

As used herein, the term “plant promoter” refers to a nucleic acid sequence capable of initiating transcription of a nucleic acid in a plant cell. The plant promoter can be derived from plants, microorganisms (such as bacteria, viruses) or animals, etc., or synthetic or engineered promoters.

As used herein, the term “Cas protein” refers to a nuclease. A preferred Cas protein is the Cas9 protein. Typical Cas9 proteins include (but are not limited to): Cas9 derived from Streptococcus pyogenes and Staphylococcus aureus. As used herein, the term “coding sequence of Cas protein” refers to a nucleotide sequence that encodes a Cas protein with cleavage activity. In the case where the inserted polynucleotide sequence is transcribed and translated to produce a functional Cas protein, the skilled person will realize that because of the degeneracy of the codon, a large number of polynucleotide sequences can encode the same polypeptide. In addition, technicians will also realize that different species have certain preferences for codons, and may optimize the codons of Cas protein according to the needs of expression in different species. These variants are specifically covered by the term “coding sequence of Cas protein”. In addition, the term specifically includes a full-length sequence that is substantially the same as the Cas gene sequence, and a sequence encoding a protein that retains the function of the Cas protein.

As used herein, the term “plant” includes whole plants, plant organs (such as leaves, stems, roots, etc.), seeds and plant cells, and their filial generation. The types of plants that can be used in the method of the present invention are not particularly limited, and generally include any higher plant types that can be transformed, including monocots, dicots and gymnospermae.

As used herein, the term “knock-in” refers to the replacement of a large fragment, especially when the replacement is a completely different sequence from the original gene.

As used herein, the term “replacement” refers to the replacement of a small fragment, a few amino acids, and a few bases.

As used herein, the term “expression cassette” refers to a polynucleotide sequence containing sequence components for the gene to be expressed and elements required for expression. The components required for expression include a promoter and a polyadenylation signal sequence. In addition, the expression cassette of the present invention may or may not contain other sequences, including (but are not limited to): enhancers, secretory signal peptide sequences and the like.

As used herein, the term “one genetic transformation” refers to: the transformants are routinely obtained through one exogenous DNA transformation and tissue culture.

As used herein, the term “two genetic transformations” refers to: firstly, a transformant or explant of targeted knock-in (NHEJ pathway) through one genetic transformation; then taking the transformant or explant obtained from the first genetic transformation as the recipient, and the targeted cleavage element is introduced to cut the repetitive sequence through the second genetic transformation, and a precise editing through the HDR pathway is achieved.

NHEJ

Non-homologous end joining (NHEJ): Without the help of any template, the ends of the double strand break are directly pulled closer to each other, and then with the help of DNA ligase, the two broken strands are rejoined.

HDR

Homologous recombination (Homology directed repair, HDR): A mechanism that relies on homologous DNA fragments for double-strand break repair in cells.

Targeted Knock-in/Replacement

A sequence is targeted knock-in/replacement at a designated site in the plant genome, that is, targeted knock-in/replacement technology, which has always been an urgently needed technology for plant research and breeding, but the existing methods are very inefficient. The present invention adopts a donor DNA fragment with a specific repeat sequence structure, after NHEJ and HDR successively, efficient targeted knock-in/replacement is achieved in plants.

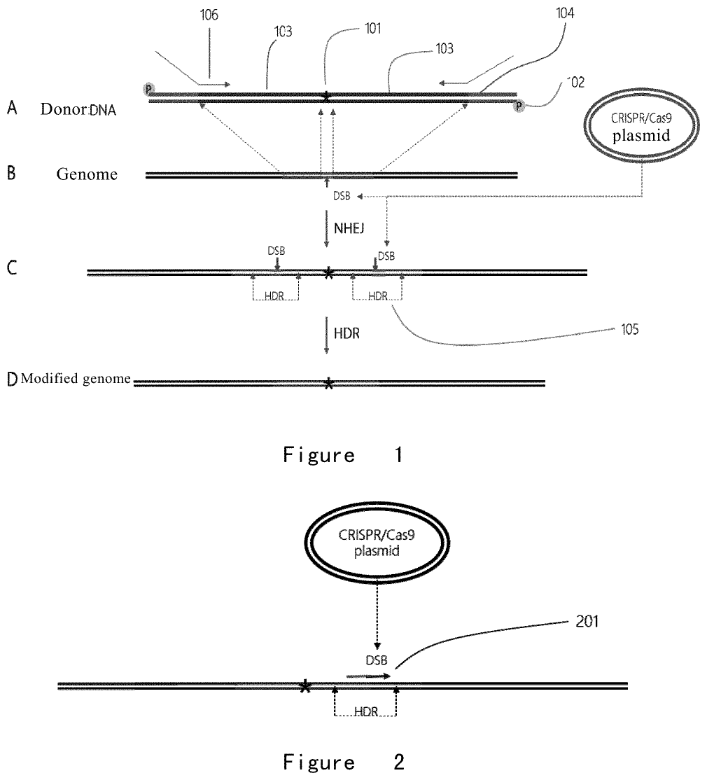

After extensive and intensive research and experimentation, the inventors have found that after NHEJ and HDR successively, DNA donors with specific repetitive sequence structures can indeed greatly improve the efficiency of targeted knock-in/replacement in plant genome editing. Therefore, the present invention aims to provide an efficient targeted knock-in/replacement method suitable for plants. As shown in FIG. 1, the implementation steps are briefly summarized as follows:

a) preparing DNA fragments in vitro for site-directed knock-in/replacement, characterized by 1) the two ends or one end of the fragment have homologous regions with the genome sequence at the position to be knocked in; 2) there is a site-directed cleavage site outside the homology region, or a site-directed cleavage site can be formed after site-specific insertion into the genome, so that the transformed cells can produce DSB; and

b) preparing DNA fragments expressing site-directed cleavage nuclease in vitro; and

c) Transforming the above two DNA fragments into a plant recipient, and under suitable conditions, making the DNA in the transformed plant cell express nuclease, and causing double-strand breaks by site-specific cleavage at the target site, so that the donor DNA fragment is integrated through NHEJ to the target site; since the donor fragment contains a sequence homologous to the target site, it can form a repeat sequence in the same direction. The presence or formation of the site-directed cleavage site between the repeat sequence can produce DSB; since the DSB between the repeat sequence can produce very efficient HDR, this feature of cells can be used to achieve site-specific precise knock-in/replacement.

In the present invention, the site-directed cleavage site can be outside the homologous sequence, or partially overlap with the homologous sequence. When the site-directed cleavage site partially overlaps with the homologous sequence, it is also considered that site-directed cleavage is located outside the homologous sequence.

Preparation of Donor DNA Fragment

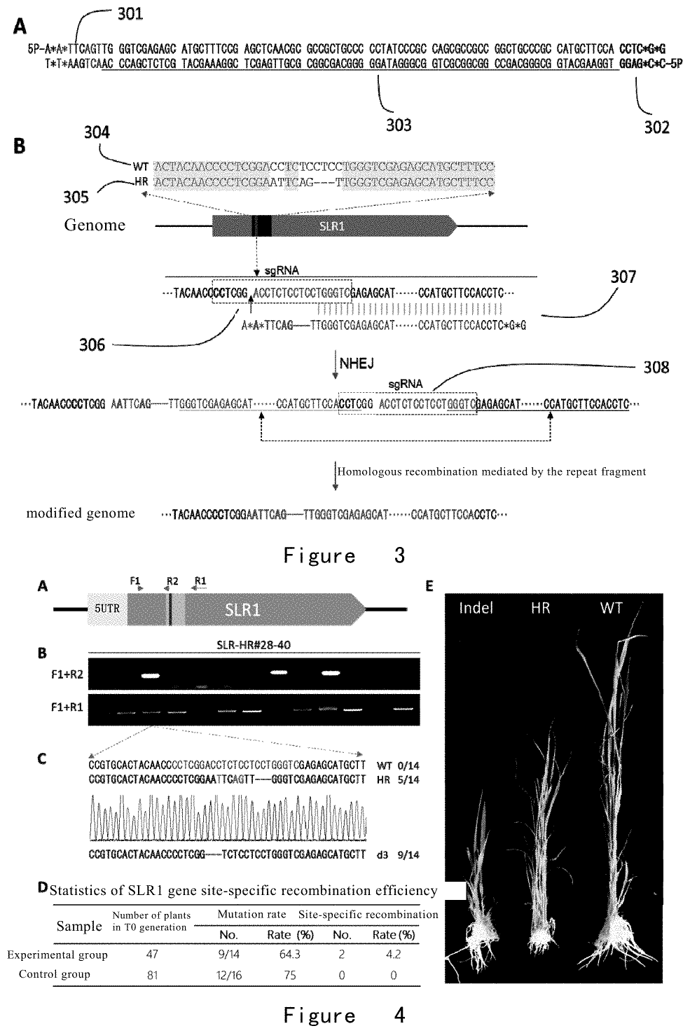

The donor DNA contains bases or fragments to be knocked in/replaced (FIG. 1A, 101), one or both sides of which are homologous fragments for HDR, the length is greater than 15 bp, preferably 20 bp-10 kb, more preferably 30 bp-5 kb, (FIG. 1, 104). The area outside the homology region that overlaps the homology region has one or more site-directed cleavage site (s), and the cleavage site (s) may be the same as the cleavage site of the genomic target or different from the cleavage site of the genomic target; the cleavage site can be completely contained in the donor DNA, or can be formed after the site-specific insertion of the donor DNA into the genome (FIG. 2, 201). In order to increase the efficiency of NHEJ, the donor DNA fragments are preferably phosphorylated at the 5′ end (FIG. 1, 102). In order to prevent the degradation of plant cell exonuclease, the terminal bases of the donor DNA fragments can be performed by sulfuration modification.

In a preferred embodiment, the preparation of the donor DNA fragment of the present invention can be carried out by the following method:

1) for the preparation of shorter donor DNA (usually within 120 bp), modified oligonucleotide single-stranded can be directly synthesized and then directly annealled to produce double-stranded donor DNA;

2) for the preparation of longer donor DNA, it can be obtained by PCR amplification using sulfuration modification primers (FIG. 1);

3) or it can be directly prepared by digesting exogenous DNA such as plasmids.

Preparation of Site-Directed Cleavage Nuclease DNA Construct

ZFN, Talen, and CRISPR/Cas9 technologies can all create double-stranded DNA breaks (DSB) by site-directed cleavage on the plant genomes. Therefore, DNA elements expressing these three site-directed cleavage nucleases can all be used in the present invention. The DNA element can be a plasmid or a linear fragment. Because the CRISPR/Cas9 technology is relatively simple and efficient, the present invention prefers CRISPR/Cas9 to make a site-specific cleavage on the plant genome.

Reagent Combination for Gene Editing

The present invention provides a reagent combination used for gene editing, the reagent combination comprises (i) a first nucleic acid construct, or a first vector containing the first nucleic acid construct, the first nucleic acid construct has a structure of Formula I from 5′-3′:

P1-A1-A2 (I)

wherein P1 is a first promoter (comprising a PolII promoter, such as 35S promoter, UBQ promoter, Actin promoter, UBI promoter, etc.);
A1 is a coding sequence of Cas9 protein;
A2 is a terminator;

and, “-” is a bond or a nucleotide linking sequence; and

(ii) a second nucleic acid construct, or a second vector containing the second nucleic acid construct, the second nucleic acid construct has a structure as shown in Formula II from 5′-3′:

P2-A3-A4-A5 (II)

wherein P2 is a second promoter (including a PolII promoter, such as tRNA promoter, 35S promoter, UBQ promoter, Actin promoter, UBI promoter, etc.);
A3 is a coding sequence of gRNA;
A4 is a transcription termination sequence (such as PolyA, PolyT, NOS terminator, UBQ terminator);
A5 is the nucleic acid construct according to the first aspect of the present invention;

and, “-” is a bond or a nucleotide linking sequence.

Various elements used in the constructs of the present invention can be obtained by conventional methods, such as PCR methods, fully artificial chemical synthesis methods, and enzyme digestion methods, and then connected together by well-known DNA ligation techniques to form the constructs of the present invention.

The vector of the present invention is transformed into plant cells so as to mediate the integration of the vector of the present invention into the chromosomes of the plant cells to prepare transgenic plant cells.

The transgenic plant cell of the present invention is regenerated into a plant body, thereby obtaining a transgenic plant.

The above-mentioned nucleic acid construct constructed by the present invention can be introduced into plant cells through conventional plant recombination technology (for example, Agrobacterium transformation technology), thereby obtaining a plant cell carrying the nucleic acid construct (or a vector containing the nucleic acid construct) is obtained, or a plant cell in which the nucleic acid construct is integrated in the genome.

Vector Construction

The main feature of this vector is to use strong promoters such as 35S, Actin or UBI to drive the expression of Cas protein in the CRISPR/Cas system, and guided by the guide RNA to the target location in the genome, and the Cas protein cuts the target, and then uses the NHEJ and HDR mechanisms to perform plant targeted knock-in or replacement.

Generally, in order to increase the activity of the protein, the proteins are usually connected by some flexible short peptides, namely Linker (connecting peptide sequence). Preferably, the Linker can use XTEN.

In order to increase knock-in and/or replacement efficiency, the present invention selects specific promoters suitable for plant cells, such as 35S, Actin or UBI promoters. Selecting the expression cassette of the guide RNA suitable for plant cells, and constructing it and the open expression cassette (ORF) of the above protein in different vectors.

In the present invention, the vector is not particularly limited, any binary vector is acceptable, not limited to pCambia vector, nor limited to these two kinds of resistance, as long as the vector that meets the following requirements can be used in the present invention: (1) It can be transformed into plants mediated by Agrobacterium; (2) it can allow RNA to be transcribed normally; (3) it can allow plants to acquire new resistance.

In a preferred embodiment, the vector is selected from the group consisting of pCambia1300, pCambia3301, pCambia2300, and a combination thereof.

Genetic Transformation

In a preferred embodiment of the present invention, the modified donor DNA fragment and the DNA fragment donor expressing the site-directed cleavage nuclease are introduced into the plant recipient. Introduction methods include, but are not limited to: gene gun method, microinjection method, electric shock method, ultrasonic method and polyethylene glycol (PEG)-mediated method, etc. Recipient plants include, but are not limited to, rice, soybean, tomato, corn, tobacco, wheat, sorghum, etc. After the above two DNA fragments are introduced into plant cells, it is speculated that precise integration can be achieved through the following steps:

1) The nuclease cuts the target site to produce DSB;

2) Donor DNA fragments are integrated to the target site through NHEJ: 5′ phosphorylation of donor DNA fragments can promote NHEJ; sulfuration modification between terminal bases can prevent the degradation of intracellular exonuclease;

3) After the site-specific integration of the donor DNA fragment, it can form a repeat sequence structure in the same direction because it contains a sequence homologous to the target site;

4) The nuclease cuts the existing or newly formed cleavage site between the repeat sequences to produce DSB;

5) Since DSB between repeat sequences can produce very efficient HDR, this feature of cells can be used to achieve site-specific and precise knock-in/replacement.

Finally, plants are obtained from site-specific recombinant cells through conventional tissue culture.

Application

The present invention can be used in the field of plant genetic engineering, for plant research and breeding, especially for genetic improvement of agricultural crops and forestry crops with economic value.

The main advantages of the present invention include:

(1) compared with the traditional direct HDR method for site-directed and precise knock-in/replacement, the donor DNA fragments with specific repeat sequences provided by the present invention can efficiently realize site-specific recombination (knock-in and/or replacement), and the efficiency of the knock-in and/or replacement is ≥12% (compared to traditional methods (only NHEJ or HDR), increased by more than 6 times), can be widely used in plant research and breeding.

(2) the donor DNA of the present invention does not need to contain a screening tag.

(3) the plant gene editing method of the present invention is simple and easy for the popularization and application.

The invention will be further illustrated with reference to the following specific examples. It is to be understood that these examples are only intended to illustrate the invention, but not to limit the scope of the invention. For the experimental methods in the following examples without particular conditions, they are performed under routine conditions (e.g. Sambrook et al., Molecular Cloning: A Laboratory Manual (New York: Cold Spring Harbor Laboratory Press, 1989) or as instructed by the manufacturer. Unless otherwise specified, all percentages, ratios, proportions or parts are by weight.

The experimental materials and reagents involved in the present invention can be obtained from commercial channels unless otherwise specified.

### Example 1 Base Substitution of Rice SLR1 Gene

Using modified DNA fragments synthesized in vitro as donor DNA fragments, combined with CRISPR/Cas9 technology, multiple bases in the rice SLR1 gene were accurately replaced and deleted. The specific operation process was as follows.

Preparation of the CRISPR/Cas9 Vector

The target gRNA-1 (SEQ ID NO.: 1) was designed for the region to be replaced in the rice SLR1 gene, and the gRNA-1 guide sequence was constructed into the CRISPR/Cas9 vector in rice. The OSU6-gRNA-1 sequence was shown in the sequence listing (SEQ ID NO.: 2).

Donor Fragment Design and In Vitro Preparation

As shown in FIGS. 3A and 3B, the end of the donor DNA can be phosphorylated and thio modified (5′P stands for 5′ end of phosphorylation modification, * stands for inter-base thio modification) to promote NHEJ; The 82 bp in the fragment is homologous to the sequence of the SLR1 target position; after the fragment is site-specific integrated, the additional 5 bases at the end (CCTCGG) and the sequence on the genome reformed the CRISPR/Cas9 target gRNA-1 to facilitate HDR.

In vitro synthesis of modified or unmodified single-stranded oligonucleotide fragments:

| | | SEQ ID | |
|---|---|---|---|
| name | Sequence (5′-3′) | NO.: | Note |
| SLR1- | 5′P-A*A*TTCAGTTGGGTCGAGAGCATGCTT | 3 | Annealing |
| UP | TCCGAGCTCAACGCGCCGCTGCCCCCTATC | | to form |
| | CCGCCAGCGCCGCCGGCTGCCCGCCATGCT | | double- |
| | TCCACCTC*G*G | | stranded |
| SLR1- | 5′P-C*C*GAGGTGGAAGCATGGCGGGCAGC | 4 | donor |
| LW | CGGCGGCGCTGGCGGGATAGGGGGCAGCG | | DNA |
| | GCGCGTTGAGCTCGGAAAGCATGCTCTCG | | fragments |
| | ACCCAACTGAA*T*T | | |
| SLR1- | CGCGGATGACGGGTTCGTGTCGCACCTGGC | 5 | Conventional |
| HR | CACGGACACCGTGCACTACAACCCCTCGG | | recombination, |
| | AATTCAGTTGGGTCGAGAGCATGCTTTCCG | | control |
| | AGCTCAACGCGCCGCTGCCCCCTATCCCGC | | |
| | C | | |

After the synthesized single-stranded oligonucleotide fragment was dissolved to 100 μM in water, it was diluted to 10 μM with annealing buffer (10 mM Tris-Cl, 0.1 mM EDTA, 50 mM NaCl, pH8.0), and annealed using the PCR machine then combined into a double-stranded donor DNA (Dn-SLR1). Among them, SLR1-HR was a single-stranded donor DNA, used as a conventional HDR experiment, as a control group.

Transformation of Rice Callus by Gene Gun

The CRISPR/Cas9 plasmid, donor DNA and gold powder was mixed according to the following system, and the rice callus pretreated with hypertonic medium for 4 hours was transformed according to the operation manual of Bole PDS-1000 desktop gene gun. Using hygromycin as a screening label, a positive resistant callus was obtained after routine tissue culture screening, which was further differentiated to obtain stable transformed plants.

| | | |
|---|---|---|
| component | concentration | volume |

| | | | |
|---|---|---|---|
| Donor DNA | 10 | uM | 1 |
| CRISPR/Cas9 Plasmid | 0.5 | ug/ul | 2 |
| Gold powder | 60 | mg/ml | 50 |

| | | |
|---|---|---|
| 2.5M Calcium chloride | 2.5M | 50 |
| spermidine | 0.1M | 20 |

Targeted Knock-in Efficiency Test

The resistant callus of the experimental group and the control group after tissue culture screening were further differentiated to obtain stable transformed plants. A total of 47 and 81 T0 generation plants were obtained in the experimental group and the control group respectively, and genomic DNA was extracted one by one for testing. Primers were designed on the upstream and downstream of the target for PCR amplification detection. The primer sequence was shown in the following table.

| | | SEQ ID | |
|---|---|---|---|
| Name | Sequence (5′-3′) | NO.: | Note |
| SLR1-F1 | GCGCTCGGGTACAAGGTG | 6 | Matched with |
| SLR1-R1 | CCAGCCTCCTGCGTGTCAA | 7 | genome |
| SLR1-R2 | GCTCTCGACCCAACTGAATT | 8 | Match with |
| | | | recombination |
| | | | sequence |

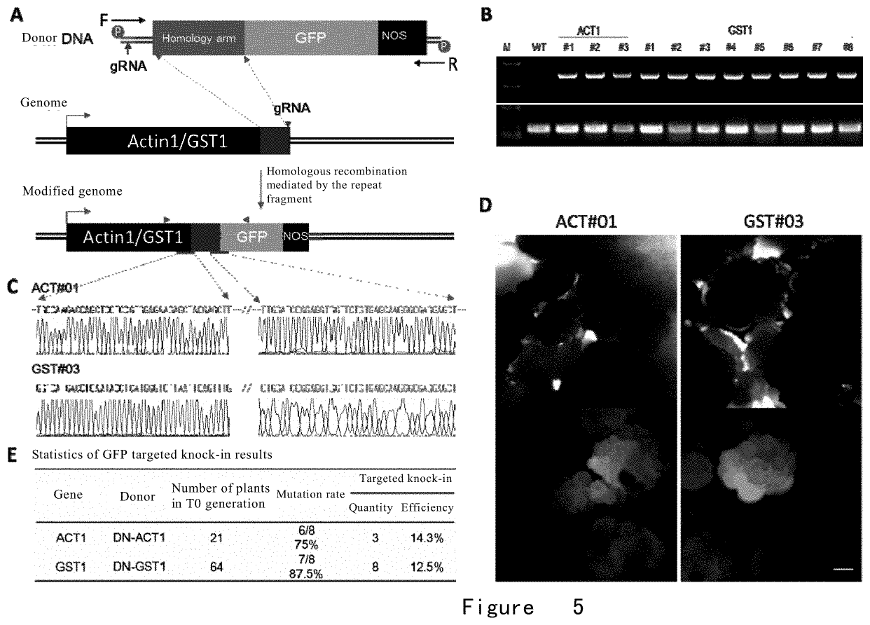

As shown in FIG. 4A, wherein SLR1-F1 and SLR1-R1 amplify genomic fragments, which serve as internal reference controls; SLR1-F1 and SLR1-R1 specifically amplify recombinant fragments to detect the efficiency of site-directed recombination. The result of electrophoresis detection after PCR amplification is shown in FIG. 4B. There are 3 samples in the experimental group that can detect specific amplified fragments, while none of the control group can be detected. Further sequencing results show that the expected site-specific replacement is occurred in 2 of the 3 positive samples (FIG. 4C), and the recombination efficiency is 4.2% (FIG. 4D). According to the experimental design, plants with precise replacement of the SLR1 site can produce a semi-dwarf phenotype, and a significant semi-dwarf phenotype can be indeed observed in the recombinant plants actually obtained in this example (FIG. 4E).

Compared with the traditional HDR experiment (control group), the experimental method based on the present invention successfully obtains the rice plant of site-specific recombination, which confirms the practical application value of the present invention.

### Example 2 Site-Specific Knock-in Experiment of GFP

Using the DNA fragments obtained by PCR amplification as donor DNA fragments, combined with CRISPR/Cas9 technology, the GFP gene was knocked into the 3′end of the high expression genes ACT1 and GST1 in rice to form a fusion protein. The specific operation process was as follows.

Preparation of CRISPR/Cas9 Vector

Target gRNA-2 and gRNA-3 (SEQ ID NO.: 9, 10) were designed for the 3′ end of the rice ACT1 and GST1 genes respectively, and these two guide sequences were constructed into the rice CRISPR/Cas9 vector, wherein OSU6-gRNA-2 and OSU6-gRNA-3 sequences were shown in the sequence listing (SEQ ID NO.: 11, 12).

Design and Preparation of Donor DNA Fragments

As shown in FIG. 5A, the donor DNA is amplified by PCR, and the primers used for amplification are shown in the table below. Among them, primers ACT1-F1 and NOS-R1 amplify the ACT1 knocked-in donor DNA fragment (sequence 9, 1528 bp); and primers GST1-F1 and NOS-R1 amplify the GST1 knocked-in donor DNA fragment (sequence 10, 1412 bp). The ends of PCR amplification primers can be phosphorylated and thio modified (5′P stands for phosphorylation modification at the 5′ end, and * stands for inter-base thio modification) to promote NHEJ; the sequence of about 400 bp in the fragment is homologous to the sequence of the target site; after the fragments are site-specific integrated into the genome, they will form a repetitive structure with the sequence of the genome; the additional target sequence of gRNA-2 or gRNA-3 at the end can be cleaved again, so that this allows HDR to occur between repeat sequences and enables precise targeted knock-in of GFP.

| | | SEQ ID | |
|---|---|---|---|
| Name | Sequence (5′-3′) | NO.: | Note |
| ACT1-F1 | 5P-C*T*ACTCGAGGGGGTGGCCCATCCATTGTGC | 13 | forward |
| GST1-F1 | 5P-G*A*TGACTACTCGAGGAGCACCTGATTGCTGGA | 14 | primer |
| NOS-R1 | 5P-G*T*GGATCCATCGTTCAAACATTTGGCAATAAA | 15 | reverse |
| | | | primer |

Transformation of Rice Callus by Gene Gun

The CRISPR/Cas9 plasmid, donor DNA and gold powder were mixed according to the following system, and the rice callus pretreated with hypertonic medium for 4 hours was transformed according to the operation manual of Bole PDS-1000 desktop gene gun. Using hygromycin as a screening label, a positive resistant callus was obtained after routine tissue culture screening, which was further differentiated to obtain stable transformed plants.

| | | |
|---|---|---|
| Component | Concentration | Volume |

| | | | |
|---|---|---|---|
| Donor DNA | 2 | ug/ul | 2 |
| CRISPR/Cas9 Plasmid | 0.5 | ug/ul | 2 |
| Gold powder | 60 | mg/ml | 50 |

| | | |
|---|---|---|
| 2.5M Calcium chloride | 2.5M | 50 |
| spermidine | 0.1M | 20 |

Detection of Targeted Knock-in Efficiency

The resistant callus of the two groups of experiments after tissue culture screening were further differentiated to obtain stable transformed plants. A total of 21 and 64 T0 generation plants were obtained in ACT1 and GST1 experiments respectively, and genomic DNA was extracted one by one for detection. Primers were designed on the upstream and downstream of the target for PCR amplification detection. The primer sequences were shown in the following table:

| | | SEQ ID | |
|---|---|---|---|
| Name | Sequence (5′-3′) | NO.: | Note |
| ACT1-F1 | TGTGAGGGACATGAAGGAGA | 16 | Matched with |
| GST1-F1 | GTACACTTGTCCTGTCTGGA | 17 | genome |
| GFP-R1 | ACGCTGAACTTGTGGCCGTT | 18 | Matched with |
| | | | recombination |
| | | | sequence |

Since ACT1 and GST1 have higher expression levels in rice, it can be judged by fluorescence whether it is integrated after fusion of GFP. Fluorescence microscopy has revealed that 3 of ACT1 and 8 of GST1 plants have significant GFP fluorescence signals (FIG. 5D). ACT1-F1+GFP-R1 and GST1-F1+GFP-R1 were used to detect the GFP integration results at these two sites respectively, and it is found that these plants with GFP fluorescence can amplify the target band (FIG. 5B). Further sequencing results show that these plants indeed have the expected targeted knock-in (FIG. 5C), and their recombination efficiencies are 14.3% and 12.5%, respectively (compared with the traditional methods using only NHEJ or HDR, they are improved by at least 7 times and 6 times, respectively) (FIG. 5E). The above experiments show that the experimental method based on the present invention successfully obtains the rice plant of the site-specific recombination, further confirming the practical application value of the present invention.

As shown in FIG. 4D, when only HDR is used, the recombination efficiency is 0, and the targeted knock-in/replacement plants cannot be obtained. The recombination efficiency of separate HDR experiments at other sites (including rice NRT1.1b gene and EPSPS gene) is also 0, and also fails to achieve targeted base replacement.

Therefore, the present invention combines NHEJ and HDR to achieve high-efficiency targeted knock-in/replacement, which is easy to implement, and has low difficulty, and can become a conventional experimental method.

All literatures mentioned in the present application are incorporated by reference herein, as though individually incorporated by reference. Additionally, it should be understood that after reading the above teaching, many variations and modifications may be made by the skilled in the art, and these equivalents also fall within the scope as defined by the appended claims.

1. A nucleic acid construct having a structure as shown in Formula I from 5′-3′:
Y1-Z1-Z2-Z3-Z4-Z5-Y2 (I)
wherein Y1 is none or a nucleotide sequence;
Z1 is a first DSB sequence;
Z2 is a first homologous sequence;
Z3 is a target DNA sequence;
Z4 is a second homologous sequence;
Z5 is a second DSB sequence;
Y2 is none or a nucleotide sequence;
and each “-” is independently a bond or a nucleotide linking sequence.

2. The nucleic acid construct of claim 1, wherein the first DSB sequence and the second DSB sequence are located (identified) and cleaved with the participation of gRNA.

3. The nucleic acid construct of claim 1, wherein each DSB sequence can be recognized and cleaved by a site-directed cleaving nuclease.

4. The nucleic acid construct of claim 1, wherein each of the DSB sequences is independently: (a) containing a cleavage site, or (b) forming a cleavage site after the nucleic acid construct is integrated into the target site by NHEJ.

5. The nucleic acid construct of claim 3, wherein the site-directed cleaving nuclease is selected from the group consisting of ZFN, Talen and CRISPR/Cas9, and a combination thereof.

6. The nucleic acid construct of claim 1, wherein the first DSB sequence, the second DSB sequence can be recognized and cleaved by an enzyme selected from the group consisting of a CRISPR-related enzyme such as Cas9, Cpf1, C2C1, C2C2, and C2C3.

7. The nucleic acid construct of claim 1, wherein the first DSB sequence, the second DSB sequence can be recognized and cleaved by an enzyme selected from the group consisting of Fok I.

8. The nucleic acid construct of claim 1, wherein the target DNA sequence is a sequence to be knocked in and/or replaced.

9. The nucleic acid construct of claim 1, wherein the nucleic acid construct is a single-stranded DNA sequence or a double-stranded DNA sequence, preferably a double-stranded DNA sequence.

10. The nucleic acid construct of claim 1, wherein the 5′ end(s) of one and/or two DNA single strand(s) of the nucleic acid construct are phosphorylated.

11. The nucleic acid construct of claim 1, wherein the phosphodiester bond between one or more (such as 2, 3, 4, or 5) bases at the end of 5′ and/or 3′ end of the nucleic acid construct is thio modified.

12. A reagent combination for gene editing, comprising:
(i) a first nucleic acid construct, or a first vector containing the first nucleic acid construct, the first nucleic acid construct has a structure of Formula I from 5′-3′:
P1-A1-A2 (I)
wherein P1 is a first promoter;
A1 is a coding sequence of Cas9 protein;
A2 is a terminator;
and, “-” is a bond or a nucleotide linking sequence; and
(ii) a donor DNA element, the donor DNA element comprises: the nucleic acid construct of claim 1, or a vector for expressing the nucleic acid construct.

13. The reagent combination of claim 12, wherein the donor DNA element comprises: a second nucleic acid construct, or a second vector containing the second nucleic acid construct.

14. The reagent combination of claim 13, wherein the second nucleic acid construct has a structure as shown in Formula II from 5′-3′:
P2-A3-A4-A5 (II)
wherein P2 is a second promoter;
A3 is a coding sequence of gRNA;
A4 is none or a transcription termination sequence;
A5 is an expression cassette of the nucleic acid construct of claim 1;
and, “-” is a bond or a nucleotide linking sequence.

15. The reagent combination of claim 12, wherein the gene editing is gene-site-directed knock-in and/or replacement.

16. A kit containing the reagent combination of claim 12.

17. A method for gene editing of a plant or plant cell, which comprises: in the presence of a donor DNA, integrating the donor DNA into a target site of the plant cell genome through NHEJ, and then DSB cleavage is performed on the sequence from the donor DNA integrated into the target site, thereby performing homologous recombination (HDR) based on the homologous sequence, thereby site-directed introducing the target DNA sequence from the donor DNA at the target site.

18. A method for gene-editing a plant or plant cell, comprising the steps:
(i) providing a plant or plant cell to be edited;
(ii) introducing a first nucleic acid construct or a first vector containing the first nucleic acid construct, and a donor DNA element comprising the nucleic acid construct of claim 1, or the vector for expressing the nucleic acid construct into the plant cell of the plant to be edited, thereby realizing the editing of the target gene of the plant or plant cell;
wherein the first nucleic acid construct has a structure of Formula I from 5′-3′:
P1-A1-A2 (I)
wherein P1 is a first promoter;
A1 is a coding sequence of Cas9 protein;
A2 is a terminator;
and, “-” is a bond or a nucleotide linking sequence.

19. A method for preparing a transgenic plant cell, comprising the steps:
(i) introducing or transfecting the nucleic acid construct of claim 1 or the reagent combination of claim 12 into a plant cell, so that the nucleic acid construct of claim 1 or the nucleic acid construct in the reagent combination of claim 12 and the chromosome in the plant cell undergo site-directed knock-in and/or replacement, thereby preparing the transgenic plant cell.

20. A method for preparing a transgenic plant cell, comprising the steps:
(i) introducing or transfecting the nucleic acid construct of claim 1 or the reagent combination of claim 12 into a plant cell, so that the plant cell contains the nucleic acid construct of claim 1 or the construct in the reagent combination of claim 12, thereby preparing the transgenic plant cell.

21. A method for preparing a transgenic plant, comprising the steps:
regenerating the transgenic plant cell prepared by the method of claim 19 or claim 20 into a plant, thereby obtaining a transgenic plant.

22. A transgenic plant cell prepared by the method of claim 19 or 20.
